(19) United States
(12) Patent Application Publication
(10) Pub. No.: US 2006/0024230 A1
(43) Pub. Date: Feb. 2, 2006

(54) Customized therapeutics and in situ diagnostics

(75) Inventors: Cynthia Bamdad, Newton, MA (US); Shoshana Bamdad Shendelman, New York, NY (US)

Correspondence Address: JHK Law, P.O. Box 1078, La Canada, CA, 91012-1078, US

(73) Assignee: Minerva Biotechnologies Corporation, Newton, MA (US)

(21) Appl. No.: 10/668,044

(22) Filed: Sep. 22, 2003

Related U.S. Application Data
(63) Continuation of application No. PCT/US02/08809, filed on Mar. 22, 2002.
(60) Provisional application No. 60/302,173, filed on Jun. 29, 2001.
(60) Provisional application No. 60/277,909, filed on Mar. 22, 2001.

(51) Int. Cl. A61K 51/00; A61M 36/14

(52) U.S. Cl. 424/1.49; 600/102

(57) ABSTRACT

Customized therapeutics and in situ diagnostics for patient therapy and diagnosis takes advantage of tools including techniques for derivatizing colloids with self-assembled monolayers. This provides the capability of a wide variety of assays including chemical or biochemical agent/agent interaction studies. Bio-derivatized colloids, with or without signaling entities, are used to probe interactions with species on non-colloidal structures. The invention provides techniques for immobilizing colloidal particles on a wide variety of non-colloidal structures. Included is the ability to decorate a variety of non-colloidal structures including beads, with colloids as a detectable assay. This allows, in many cases, assays detectable via the unaided human eye, as well as assays detectable via automated determination of a change of interaction of electromagnetic radiation with the colloids, e.g., absorption, light-scattering, and the like.

### RELATED APPLICATIONS

This application is a continuation of International Application No. PCT/US02/08809 filed Mar. 22, 2002, which was published under PCT Article 21(2) in English, and claims priority to U.S. provisional application Ser. No. 60/302,173, filed Jun. 29, 2001, and U.S. provisional application Ser. No. 60/277,909, filed Mar. 22, 2001, each of which is incorporated by reference herein.

### FIELD OF THE INVENTION

This invention relates generally to assays and techniques for diagnosing or detecting a disease state or a susceptibility to a disease state of a patient and/or selecting or determining a particular therapeutic protocol for treatment or prevention of the disease state based on the efficacy of the protocol for a specific individual patient or patient class. Techniques including drug screening of drugs known to treat a particular disease in order to select a particular drug most efficacious for treating a specific individual patient or patient class and detection of targets associated with specific disease states in a surgical operating field or internally of a patient are facilitated by the invention.

### BACKGROUND OF THE INVENTION

Currently drug treatments and other treatment protocols for treating a particular medical condition or disease state are typically chosen based their effects in large-scale standardized screening experiments and based on tests conducted on a relatively small group of test patients in clinical trials. However, person-to-person genetic variations affect a drug's or treatment protocol's efficacy against a disease or medical condition in an individual, and also can effect the effective dosage, toxicity and side effects of the drug or treatment protocol. One reason for such individualized response to drug therapy is due to inherent genetic differences between individuals affecting protein expression and/or structure by cells within the body. In addition, individuals with certain diseases such as cancer may no longer express a standard protein repertoire, and their disease-modulated repertoire can continuously change as the disease progresses. Each of the above variations in the protein expression/structure repertoire can affect the efficacy of a drug treatment or other treatment protocol for a particular, individual and point to a need for testing of a treatment protocol for efficacy in treating a particular individual.

Typically employed analytical drug screening techniques suffer from one or more short comings (e.g. they are time-consuming, cumbersome, difficult to perform, etc.) making them poorly suited for use in performing individualized drug/treatment protocol assays for customized therapeutics. What is needed are new high-throughput screening methods that can involve the use of discrete molecular targets for screening to render feasible screening tests focused on efficacy of a drug and/or treatment protocol in treating an individual patient (or patient group with similar genetic variations). In certain embodiments, the present invention provides such techniques.

In the context of typical surgical procedures involving the removal of undesirable cells and/or tissue (e.g. cancerous cells) from the body of a patient, standard practice currently requires that the surgeon makes a best guess as to how much tissue should be removed. In typical state-of-the-art techniques, a sample is first excised from the patient, and then the excised sample is sent to a centralized laboratory and after some period of time, the specimen is analyzed. Of special interest are the boundary areas. If a margin of the excised sample is found to be positive for some medical condition such as cancer, then the surgeon must re-excise. This practice puts the patient at risk by imposing several time delays during which cancer cells may break free and spawn distant metastases. Additionally, the surgeon is under pressure to remove more rather than less, which may not be best in all cases.

In one known procedure for removing facial cancers (“Mohs” surgery—a special procedure in which the cancer is shaved off one layer at a time), analysis of an excised sample occurs during the surgical procedure itself. This procedure employs standard techniques of making frozen tissue sections and analyzing them with existing methods and miniaturized equipment located in the operating room. The surgeon shaves away the tumor and each piece removed is frozen, sectioned and analyzed in the operating room. While this technique has the advantage of occurring during the actual surgical procedure, it requires the use of specialized equipment, which must be operated by highly trained individuals, is still time consuming and difficult to employ, and is only able to determine whether excised tissue is free of cancer, not whether the tissue remaining with the patient is free of cancer, which is most critical. Thus, as above, to be conservative, the surgeon is under pressure to remove more rather than less, which may not be best in all cases. Mohs surgery is performed only by specially trained physicians and often requires a reconstructive procedure as follow-up.

What is needed are fast, easy to perform, and highly specific diagnostic techniques and assays that can be performed in situ on or within the body of a patient undergoing a surgical procedure able to detect the presence of undesirable tissue or cells (e.g. cancerous cells) remaining behind after excision of cells or tissue. Certain embodiments of the present invention provide such techniques and assays.

### SUMMARY OF THE INVENTION

Typical current state of the art binding assays (e.g. sandwich assays, immunoprecipitation and the like) are too time-consuming and cumbersome to be used effectively for customized screening of drugs and treatment protocols for individual patients (or groups of patients with similar genetic variations affecting response to a treatment protocol) or in situ hystopathology in an intraoperative procedure. Additionally, typical existing assays must be performed by personnel specifically trained in biochemistry. Methods of some embodiments the invention make use of novel and modular intermediates such as colloid particles (i.e. nanoparticles) that can readily be used and analyzed by unskilled personnel or by persons such as surgeons whose expertise is in a different field.

Certain methods of the invention describe technology that makes assessment of the efficacy of a drug agent or other treatment protocol for a particular individual feasible. Using such methods it is feasible to perform, for example, a focused drug screen to identify drugs that are optimally suited to treat a certain individual for a certain medical condition. Until now, issues of cost, time and effort prohibited the process of identifying customized therapeutics.

In one embodiment, the invention involves a method, comprising steps of exposing at least a portion of a biological sample derived from a patient indicated for treatment for, or at risk of acquiring, a medical condition to a first therapeutic protocol known to have efficacy for treating or preventing the condition, and determining a response of the biological sample indicative of the effectiveness of the first therapeutic protocol in treatment or prevention of the condition.

In another embodiment, the invention involves a method comprising steps of exposing a biological sample, taken from a patient indicated for treatment for a medical condition, to a therapeutic protocol, determining the effectiveness of the protocol in treatment of the medical condition, and treating the patient according to the protocol.

Also described are methods to sensitively and accurately perform pathology and disease profiling in situ, on or within a body of a patient, for example during surgical procedures. In one such embodiment, in an intraoperative procedure, remaining tissues are exposed to intermediates such as colloids that can bear, or to which can become immobilized, signaling entities and molecular probes comprising chemical or biological binding partners that can bind to biological species that are indicative of a specific disease state. Unbound colloids can be washed away and the accumulation of colloids in the affected area is detected. This in situ hystopathology can greatly improve patient outcome by improving the accuracy of surgical procedures. The technology can provide the surgeon with a “molecular blueprint” of the tissue, so that all the affected areas can be removed without unnecessarily radical procedures.

In this and some other embodiments of the invention, one important feature is the ability to cause a chemical or biological binding partner, or probe, to bind to biological species at a selected area of tissue in a manner that is detectable. One way that this can be carried out is to provide an intermediate entity to which both the binding partner and signaling entity are fastened or to which both can become immobilized, and to expose the binding partner, intermediate, and signaling entity to the tissue and to determine immobilization of the signaling entity relative to the tissue. The signaling entity and binding partner can be immobilized relative to the intermediate in any order, and each can be immobilized relative to the intermediate before, during, or after binding of the binding partner to biological species at the tissue surface. For example, an intermediate carrying both an immobilized binding partner and an immobilized signaling entity can be exposed to a selected area of tissue, binding of the binding partner to biological species at the tissue surface can be allowed to occur, unbound binding partner/intermediate/signaling entity can optionally be washed away, and signaling entity at the selected area of tissue surface can be determined (the presence and/or amount of signaling entity can be observed). As another example, the selected area of tissue can be exposed individually to signaling entity, intermediate, and binding partner (in any order), and if the binding partner binds to biological species at the tissue surface than by virtue of binding of both binding partner and signaling entity to intermediate, signaling entity will be immobilized relative to the selected tissue surface area. The intermediate can comprise any entity to which at least a binding partner, and in some embodiments both a signaling entity and the binding partner, can become immobilized. Examples include particulate material such as colloids, molecules such as polymer molecules, dendrimers, and even ionic, covalent, coordinative, or other chemical bonds (e.g. the signaling entity can be directly bound to the binding partner). In all embodiments of the invention utilizing a colloid particle; particle-like structure, any intermediate can be used in place of the colloid particle. In some other embodiments, the intermediate (e.g. a colloid particle, quantum dot, etc.) may itself act as the signaling entity.

The above inventive techniques, in some embodiments, differ from known techniques in that: 1) typical known techniques analyze sections removed whereas such embodiments of the present invention involve analysis of sections that remain; 2) such embodiments of the present invention can employ precise molecular probes rather than relying on bulk properties like cell morphology, so that with the present techniques, one can detect a cell's propensity to become cancerous and also get a molecular assessment of the patient's prognosis; 3) such embodiments of the present invention can employ technology that is portable, easy to use by unskilled personnel, and able to be used in the context of a wide variety of medical conditions and procedures.

In one embodiment, the invention involves a method comprising the steps of applying to a localized region of a body of a patient a binding partner immobilized relative to or able to be immobilized relative to a signaling entity, and determining immobilization of the signaling entity within or on the body of the patient.

In another embodiment, the invention involves method comprising the steps of applying to a body of a patient a binding partner immobilized relative to or able to be immobilized relative to a signaling entity, wherein the signaling entity is able to enhance an optical contrast of a tissue of the body to which it becomes immobilized, optically determining immobilization of the signaling entity to the tissue.

In yet another embodiment, the invention involves a method involving comprising promoting the application to a localized region of a body of a patient of a binding partner immobilized relative to or able to be immobilized relative to a signaling entity, and the determination of immobilization of the signaling entity within or on the body of the patient.

In another embodiment, the invention involves a method comprising promoting the application to a localized region of a body of a patient of a colloid particle, and the determination of fastening of the colloid particle within or on the body of the patient.

In one embodiment, the invention involves a method comprising the steps of allowing a colloid particle the ability to fasten to a localized region of a body of a patient, and determining fastening of the colloid particle within or on the body of the patient.

In another embodiment, the invention involves a method comprising the steps of allowing a colloid particle the ability to fasten to a body of a patient, wherein the colloid particle is capable of enhancing the optical contrast of a tissue of the body to which it becomes fastened, and optically determining fastening of the colloid particle to the tissue.

In yet another embodiment, the invention involves a method comprising steps of allowing a colloid particle the ability to fasten to a cell taken from a patient indicated for treatment for a medical condition, and determining fastening of the colloid particle to the cell.

In another aspect, the present invention involves a series of kits. In one embodiment, the invention involves a kit comprising at least one of a signaling entity, and a binding partner capable of being immobilized relative to the signaling entity. The kit also comprises instructions directing a user to immobilize the signaling entity relative to the binding partner, to apply the binding partner and signaling entity to a localized region of a body of a patient, and to determine immobilization of the signaling entity within or on the body of the patient.

In another embodiment, the invention involves a kit comprising an article comprising a signaling entity, and a binding partner immobilized relative to the signaling entity. The kit further comprises instructions directing a user to apply the article to a localized region of a body of a patient, and to determine immobilization of the article within or on the body of the patient.

In yet another embodiment, the invention involves a kit, comprising a colloid particle. The kit also comprises instructions directing a user to apply the colloid particle to a localized region of a body of a patient, and to determine fastening of the colloid particle within or on the body of the patient.

The invention also provides a variety of methods, compositions and species, and articles for monitoring (detecting) interactions between chemical or biological species including techniques useful for drug screening that can be useful for practicing the present invention. Also, in this context, cell studies, especially techniques involving interactions between ligands and cell surface proteins and receptors are disclosed. Discovery and therapeutics involving drugs that can affect these interactions also is described, with an emphasis on drug screening to identify drugs or therapeutic protocols most suited for treating a specific individual patient (or group of patients with similar genetic variations affecting response to a drug treatment protocol). Another area involves detecting proteins, either in solution or on the surfaces of intact cells, for diagnostic purposes, for example for detecting cell surface proteins on cells within an operating field of a patient undergoing a surgical procedure.

The disclosure also describes techniques and components that allow for a method of signaling a single binding of a first biological or chemical agent to a second biological or chemical agent with a plurality of signaling entities useful for performing certain aspects of the current invention. An article facilitating this technique and defining a first biological or chemical agent capable of biological or chemical binding to a second agent, linked to a plurality of signaling entities is disclosed. The plurality of signaling entities can be linked to the agent by way of a polymer or dendrimer that carries a plurality of signaling entities and is adapted for linkage to an agent. The technique also can be facilitated by immobilizing the agent at a colloid particle, e.g. at the surface of a colloid particle at which a plurality of signaling entities also are, or can be, immobilized. In preferred embodiments, more than three signaling entities signal a single chemical or biological binding event simultaneously. More preferably, at least 10, more preferably at least 50, and more preferably at least 1000 or 10,000 signaling entities signal a single chemical or biological binding event simultaneously according to this aspect of the invention.

Also disclosed herein is a series of components and techniques for performing drug screening useful for practicing certain embodiments of the invention. The approach in certain embodiments provides 1) a modular system for the attachment of natural ligands to universal signaling elements; 2) enhanced sensitivity of detection through the attachment of a plurality of signaling elements to each ligand; 3) a simpler format (without the need for washing steps, enzymatic cleavage and toxic substrates); 4) the capability for multiplexing.

It is not intended that the present invention be limited by the nature of any solid support utilized to carry ligands and/or binding partners and/or signaling entities. In one embodiment, the solid support is a colloid (e.g. gold colloid). In another embodiment, the solid support is a semiconductor nanocrystal (i.e. a “Quantum dot,” as disclosed, for example, in U.S. Pat. Nos. 6,207,392 and 5,990,479). A very wide variety of additional solid surfaces an materials can potentially act as the solid support. For example, a non-limiting list of articles that can be modified to comprise a solid support according to the invention include an electrode, a silicon chip, a glass slide, a surface of a probe or sensor, a plastic tissue culture plate or dish or well or flask, a polymeric bead that may or may not be magnetic, a wall of a tube, a flow channel, etc.

It is also not intended that the present invention be limited by the nature of attachment of the ligand to the solid support. In one embodiment, said ligand is covalently attached (directly or through another ligand or binding moiety) to the solid support. In another embodiment, the ligand is attached non-covalently or by electrostatic or ionic interaction. It is also not intended that the present invention be limited by the timing of when a ligand or binding partner is attached to the solid support. In one embodiment, a portion of a surgical field is incubated with a cognate ligand that carries an affinity tag. After washing the area to remove unbound ligands, signaling colloids bearing binding partners of the affinity tags are added.

The present invention can utilize a variety of signaling elements, including but not limited to fluorescent molecules and enzymes capable of acting on color-producing substrates. In one embodiment, the present invention utilizes electroactive molecules that is, molecules having an oxidation/reduction potential that can be determined electronically or electrochemically proximate a working electrode of an appropriate, conventional electrical arrangement, as signaling elements. Examples of suitable electroactive molecules can be found in Applicant's International Patent Publication No. WO 01/92277, incorporated herein by reference). In certain other embodiments, colloids act as signaling elements by their ability to induce a color change in a solution or their ability to increase the visual contrast of a substrate upon aggregation indicative of binding to a substrate and/or other colloid particles. In yet other embodiments, quantum dots act as signaling elements.

It is not intended that the present invention be limited by the nature of chemical or biochemical agents. A wide variety of agents and binding partners of those agents such as protein/protein, protein/peptide, antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc., can potentially be used for certain binding interactions of the inventions. In one embodiment, the agent is a ligand, specifically a peptide. In a preferred embodiment, the peptide is derivatized with a moiety (such as a histidine tag) that can bind to a metal chelate. In this embodiment, it is convenient that the solid support comprise a metal chelate and said peptide is attached to said solid support via binding of said moiety to said metal chelate.

In some embodiments of techniques useful for practicing certain embodiments of the invention, molecules, such as cell-derived molecules including both cell-surface receptors and intracellular signaling proteins, exist on or are attached to solid supports that can either be surfaces or particle-like in nature. Binding partners of these molecules, which can include putative drug candidates or known drug agents, are attached to surfaces and/or particle-like structures, and are allowed to interact with the cell-derived proteins in a manner such that binding between the two binding partners occurs. One of the binding partners or its attached support can additionally be derivatized with a detectable substance. Interacting complexes are identified using characteristics of the associated complex that differentiate it from the unassociated binding partners. The presence of, or a change in, a detectable moiety, that is either co-immobilized with one of the binding partners on a common solid support or directly attached to one of the binding partners, is detected. Molecules that disrupt a relevant interaction can be identified by detecting a loss of this signal.

For example, a chemical or biological agent can be attached to the surface of a solid support. A binding partner of the chemical or biological agent can be attached to a colloid, and exposure of the colloid to the surface of the article results in binding of the binding partner to the agent, thus immobilization of the colloid at the surface. This can be determined by determination of the colloid at the surface, for example via an auxiliary signaling entity immobilized with respect to the colloid particle that facilitates this determination (such as an electroactive signaling entity or a visual signaling entity such as a fluorescent tag).

In this and other similar embodiments of the invention, immobilization of various entities, e.g. binding partner and/or signaling entity, to the colloid can take place in any order, and each can take place before or after binding of the binding partner to the biological or chemical agent.

In certain drug screening techniques useful for practicing certain embodiments of the invention, cell-derived molecules, such as proteins are attached to surfaces (e.g. cell surfaces, particle surfaces, substrates, etc.) and ligands, as well as putative drug candidates are attached to particles, and are allowed to interact with the cell-derived proteins in a manner such that binding between the two binding partners occurs. One of the binding partners or its attached support can be derivatized with a detectable substance.

For example, according to one embodiment of the present invention, samples comprising cells and/or biological molecules such as proteins, peptides, nucleic acids, etc. derived from a patient are separately treated with a panel of drugs. To determine the therapeutic effect of the drug on that particular patient's sample indicative of the therapeutic effect of the drug on that particular patient, assays of the invention are used to detect, measure or determine a pattern of cell surface receptors and/or binding interactions that are indicators of the disease state. By performing the assay before and after drug treatment of the sample, the drug's efficacy is determined. Many cancer cells display proteins on their surfaces that are cancer markers and also can be measured to assess disease state as well as prognosis. Many human cancers are characterized by the aberrant expression of the MUC 1 receptor. In a specific example, MUC 1 positive tumor cells are drawn from a patient then expanded in culture. Aliquots of the growing cells are then tested with a panel of drugs. Following drug treatment, the cells are probed with colloids that present both a fluorescent signaling moiety and an antibody against a tumor-specific portion of the receptor. The level of measured MUC 1 receptor on the cell surface as well as the location and accessibility of the receptor indicate efficacy of the drugs tested. Low levels or absence of receptor expression as well as receptor clustering at the apical border are associated with healthy cells, while high expression levels and loss of clustering and loss of an interchain binding region (see Applicant's co-pending U.S. patent application Ser. No. 09/996,069, filed Nov. 27, 2001, entitled “Diagnostic Tumor Markers, Drug Screening for Tumorigenesis Inhibition, and Compositions and Methods for Treatment of Cancer (MUC 1)”) are indicators of cancer. Tissues exposed during a surgical procedure can be similarly tested.

In another aspect, the invention comprises methods of making any of the embodiments described herein. In still another aspect, the invention comprises methods of using any of the embodiments described herein

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is typically represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIG. 1 shows cells bearing a tumor marker MUC-1 that are incubated with electronic signaling colloids that present the MUC-1 specific antibody, DF-3. The antibody is attached to the colloid via a His-tagged protein G that is bound to NTA/Ni(II) groups that were incorporated into a self-assembled monolayer on the colloid.

FIG. 2 shows a tissue specimen that is attached to a flexible, semi-permeable support via interaction with peptides containing RGD motifs. The specimen is then incubated with electro-active signaling colloids that bear a ligand for a cell-surface receptor of interest. The specimen is rinsed, then interfaced with a microelectrode array. The electrode array can be made such that the electrode dimensions are comparable to cell dimensions. The specimen is then characterized by ACV, which can then be correlated to histopathology.

FIG. 3 shows ACV demonstration of enhanced electronic communication across a self-assembled monolayer, and redox signaling of protein immobilization to a cell surface, against a control.

FIGS. 4A and 4B are photocopies of photomicrographs (40× magnification) of cells decorated by colloids selectively at locations where receptor was expressed (4B) and control (4A).

### DETAILED DESCRIPTION OF THE INVENTION

The following International Patent Applications and International Publications and co-pending, commonly owned U.S. patent applications describe in detail many methods and materials useful in performing certain binding, diagnostic, and drug screening assays of the context of the present invention. Certain of these methods and materials are also disclosed and/or referred to and/or incorporated herein below, particularly in the disclosure below preceding the section heading entitled “Customized Therapeutics and In Situ Diagnostics”. Greater detail and additional materials and methods useful or potentially useful in the context of the present invention can be found in the applications and publications listed below. International patent application serial number PCT/US00/01997, filed Jan. 25, 2000 by Bamdad et al., entitled “Rapid and Sensitive Detection of Aberrant Protein Aggregation in Neurodegenerative Diseases” (published as international patent publication no. WO 00/43791 on Jul. 27, 2000), International patent application serial number PCT/US00/01504, filed Jan. 21, 2000 by Bamdad, et al, entitled “Interaction of Colloid-Immobilized Species with Species on Non-Colloidal Structures” (published as international patent publication no. WO 00/43783 on Jul. 27, 2000), International patent application serial number PCT/US01/20232, filed Jun. 25, 2001 by Bamdad et al., entitled “Rapid and Sensitive Detection of Aberrant Protein Aggregation” (published as international patent publication no. WO 02/01230 on Jan. 3, 2002), International patent application serial number PCT/US01/44783, filed Nov. 27, 2001 by Bamdad, et al, entitled “Diagnostic Tumor Markers, Drug Screening for Tumorigenesis Inhibition, and Compositions and Methods for Treatment of Cancer (MUC 1)”, commonly-owned, copending U.S. patent application Ser. No. 09/602,778, filed Jun. 23, 2000 by Bamdad et al., entitled “Interaction of Colloid-Immobilized Species with Species on Non-Colloidal Structures”; commonly-owned, copending U.S. patent application Ser. No. 09/631,818, filed Aug. 3, 2000 by Bamdad et al., entitled “Rapid and Sensitive Detection of Protein Aggregation”; commonly-owned, copending U.S. patent application Ser. No. 09/996,069, filed Nov. 27, 2001, by Bamdad et al. entitled “Diagnostic Tumor Markers, Drug Screening for Tumorigenesis Inhibition, and Compositions and Methods for Treatment of Cancer (MUC 1)”, all are incorporated herein by reference.

Definitions:

“Small molecule”, as used herein, means a molecule less than 5 kiloDalton, more typically less than 1 kiloDalton. As used herein, “small molecule” excludes proteins.

The term “candidate drug” as used herein, refers to any medicinal substance used in humans, animals, or plants. Encompassed within this definition are compound analogs, naturally occurring, synthetic and recombinant pharmaceuticals, hormones, antimicrobials, neurotransmitters, etc. This includes any substance or precursor (whether naturally occurring, synthetic or recombinant) which is to be evaluated for use as a drug for treatment of a particular disease desired to be treated or prevention thereof. Evaluation typically takes place through activity in an assay, such as the screening assays disclosed herein.

A variety of types of particles can be used in the invention. For example, “fluid suspendable particle” means a particle that can be made to stay in suspension in a fluid in which it is used for purposes of the invention (typically an aqueous solution) by itself, or can be maintained in solution by application of a magnetic field, an electromagnetic field, agitation such as stirring, shaking, vibrating, sonicating, centrifuging, vortexing, or the like. A “magnetically suspendable” particle is one that can be maintained in suspension in a fluid via application of a magnetic field. An electromagnetically-suspendable particle is one that can be maintained in suspension in a fluid by application of an electromagnetic field (e.g., a particle carrying a charge, or a particle modified to carry a charge). A “self-suspendable particle” is a particle that is of low enough size and/or mass that it will remain in suspension in a fluid in which it is used (typically an aqueous solution), without assistance of for example a magnetic field, for at least 1 hour. Other self-suspendable particles will remain in suspension, without assistance, for 5 hours, 1 day, 1 week, or even 1 month, in accordance with the invention.

“Proteins” and “peptides” are well-known terms in the art, and are not precisely defined in the art in terms of the number of amino acids that each includes. As used herein, these terms are given their ordinary meaning in the art. Generally, peptides are amino acid sequences of less than about 100 amino acids in length, but can include sequences of up to 300 amino acids. Proteins generally are considered to be molecules of at least 100 amino acids.

As used herein, a “metal binding tag” refers to a group of molecules that can become fastened to a metal that is coordinated by a chelate. Suitable groups of such molecules include amino acid sequences including, but not limited to, histidines and cysteines (“polyamino acid tags”). Metal binding tags include histidine tags, defined below.

As used herein, “chelate coordinating a metal” or metal coordinated by a chelate, refers to a metal coordinated by a chelating agent that does not fill all available coordination sites on the metal, leaving some coordination sites available for binding via a metal binding tag.

As used herein, “metal binding tag/metal/chelate linkage” defines a linkage between first and second species in which a first species is immobilized relative to a metal binding tag and a second species is immobilized relative to a chelate, where the chelate coordinates a metal to which the metal binding tag is also coordinated. U.S. Pat. No. 5,620,850 of Bamdad, et al., incorporated herein by reference, describes exemplary linkages.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., colloid particles), entities that absorb or emit electromagnetic radiation at a level or within a wavelength range such that they can be readily detected visibly (unaided or with a microscope including an electron microscope or the like), or spectroscopically, entities that can be detected electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition, phosphorescent moieties), up-regulating phosphors, chemiluminescent entities, electrochemiluminescent entities, or enzyme-linked signaling moieties including horse radish peroxidase and alkaline phosphatase, naturally fluorescent proteins, and particles made up of material(s) that can emit or can be induced to emit a detectable signal (e.g. semiconductor nanocrystal “Quantum dots” as described, for example, in U.S. Pat. Nos. 6,207,392 and 5,990,479) “Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as protein A, which is covalently attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) which molecule also presents a chelate coordinating a metal. A species also is adapted to be fastened to a surface if a surface carries a particular nucleotide sequence, and the species includes a complementary nucleotide sequence.

“Covalently fastened” means fastened via nothing other than one or more covalent bonds. E.g. a species that is covalently coupled, via EDC/NHS chemistry, to a carboxylate-presenting alkyl thiol which is in turn fastened to a gold surface, is covalently fastened to that surface.

As used herein, a component that is “immobilized relative to” another component either is fastened to the other component or is indirectly fastened to the other component, e.g., by being fastened to a third component to which the other component also is fastened, or otherwise is translationally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc. All entities that can be fastened or adapted to be fastened to other entities of the invention also can be immobilized or adapted to be immobilized to the other entities, and vice versa.

“Specifically fastened” or “adapted to be specifically fastened” means a species is chemically or biochemically linked to another specimen or to a surface as described above with respect to the definition of “fastened to or adapted to be fastened”, but excluding all non-specific binding.

“Non-specific binding”, as used herein, is given its ordinary meaning in the field of biochemistry.

“Colloids”, as used herein, means nanoparticles, i.e. very small, self-suspendable or fluid-suspendable particles including those made of material that is, e.g., inorganic or organic, polymeric, ceramic, semiconductor, metallic (e.g. gold), non-metallic, crystalline, amorphous, or a combination. Typically, colloid particles used in accordance with the invention are of less than 250 nm cross section in any dimension, more typically less than 100 nm cross section in any dimension, and in most cases are of about 2-30 nm cross section. One class of colloids suitable for use in the invention is 10-30 nm in cross section, and another about 2-10 nm in cross section. As used herein this term includes the definition commonly used in the field of biochemistry.

A “moiety that can coordinate a metal”, as used herein, means any molecule that can occupy at least two coordination sites on a metal atom, such as a metal binding tag or a chelate.

“Diverse biological species” means different animals, such as mouse and hamster, mouse and goat, etc.

The term “sample” refers to any cell, tissue, or fluid from a biological source (a “biological sample”), or any other medium, biological or non-biological, that can advantageously be evaluated in accordance with the invention including, but not limited to, a biological sample derived from a human patient, or an animal, or the like. A sample “derived from” a patient/animal etc., as used herein refers both to samples drawn from such patient/animal, as well as samples prepared from a precursor sample drawn from such patient/animal, for example through modification, extraction, purification, expansion, transformation, expression, partition, etc. of the precursor sample. A sample “derived from” a patient/animal also encompasses a sample that is completely synthetic and is not prepared from any sample or other material ever drawn from a patient/animal, but rather is synthesized based on information about the patient/animal to contain at least one component or species that mimics or is essentially equivalent to a biological molecule (e.g. a protein, peptide, nucleic acid, etc.) of the particular individual patient/animal (or group of patients/animals with similar genetic variations related to the particular characteristics of the biological molecule). One example of a sample derived from a patient is a sample drawn from a human or animal, for example, to whom a candidate drug has been given, or is proposed to be given, to determine the efficacy of the drug. Another example of a sample derived from a patient is a plurality of cells that have been expanded (and, optionally immortalized, fused with other cells, etc.) in culture from a sample of cells initially drawn from the patient. Another example of a sample derived from a patient is a solution including a purely synthetic biological molecule or portion thereof that is essentially identical to a corresponding biological molecule or portion thereof of the patient. A sample derived from a patient can also refer to a cell or tissue sample located on or within a body of a patient, for example a tissue/cell sample in a surgical field of a patient.

A “sample suspected of containing” a particular component means a sample with respect to which the content of the component is unknown. For example, a fluid sample from a human suspected of having a disease, but not known to have the disease, defines a sample suspected of containing a disease associated species. “Sample” in this context includes naturally-occurring samples, such as physiological samples from humans or other animals, samples from food, livestock feed, etc., as well as “structurally predetermined samples”, which are defined herein to mean samples, the chemical or biological sequence or structure of which is a predetermined structure used in an assay designed to test whether the structure is associated with a particular process such as a disease state. As alluded to above, such structurally predetermined sample can be derived from a patient in certain embodiments. For example, a “structurally predetermined sample” includes a peptide sequence, random peptide sequence in a phage display library, and the like. For example, some typical samples drawn from humans or other animals include cells, blood, urine, ocular fluid, saliva, cerebro-spinal fluid, fluid or other samples from tonsils, lymph nodes, needle biopsies, etc.

As used herein, a “metal binding tag” refers to a group of molecules that can become fastened to a metal that is coordinated by a chelate. Suitable groups of such molecules include amino acid sequences, typically from about 2 to about 10 amino acid residues. These include, but are not limited to, histidines and cysteines (“polyamino acid tags”). Such binding tags, when they include histidine, can be referred to as a “poly-histidine tract” or “histidine tag” or “HIS-tag”, and can be present at either the amino- or carboxy-terminus, or at any exposed region, of a peptide or protein or nucleic acid. A poly-histidine tract of six to ten residues is preferred for use in the invention. The poly-histidine tract is also defined functionally as being a number of consecutive histidine residues added to a protein of interest which allows the affinity purification of the resulting protein on a metal chelate column, or the identification of a protein terminus through the interaction with another molecule (e.g. an antibody reactive with the HIS-tag).

“Affinity tag” is given its ordinary meaning in the art. Affinity tags include, for example, metal binding tags, GST (in GST/glutathione binding clip), and streptavidin (in biotin/streptavidin binding). At various locations herein specific affinity tags are described in connection with binding interactions. It is to be understood that the invention involves, in any embodiment employing an affinity tag, a series of individual embodiments each involving selection of any of the affinity tags described herein.

“Molecular wires” as used herein, means wires that enhance the ability for a fluid encountering a SAM-coated electrode to communicate electrically with the electrode. This includes conductive molecules or, as mentioned above molecules that can cause defects in the SAM allowing communication with the electrode. A more detailed discussion of molecular wires as well as exemplary molecules useful for forming them can be found in international patent publication nos. WO 00/43783, WO 02/01230 and WO 00/43791, all previously incorporated by reference.

The term “binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “binding partner” refers to a molecule that can undergo binding with a particular molecule. Biological binding partners are examples. For example, Protein A is a binding partner of the biological molecule IgG, and vice versa.

A “ligand” to a cell surface receptor, refers to any substance that can interact with the receptor to temporarily or permanently alter its structure and/or function. Examples include, but are not limited to binding partners of the receptor and agents able to alter the chemical structure of the receptor (e.g. modifying enzymes).

The term “determining” refers to quantitative or qualitative analysis of a species via, for example, spectroscopy, ellipsometry, piezoelectric measurement, immunoassay, electrochemical measurement, visual or optical observation and the like. “Determining” also means detecting or quantifying interaction between species, e.g. detection of binding between two species.

The term “self-assembled monolayer” (SAM) refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. See Laibinis, P. E.; Hickman, J.; Wrighton, M. S.; Whitesides, G. M. Science 245, 845 (1989), Bain, C.; Evall, J.; Whitesides, G. M. J. Am. Chem. Soc. 111, 7155-7164 (1989), Bain, C.; Whitesides, G. M. J. Am. Chem. Soc. 111, 7164-7175 (1989), each of which is incorporated herein by reference. A wide variety of SAMs can be used in accordance with the invention, on a wide variety of surfaces, to present desired species such as binding partners, signaling entities, and the like at a surface of an article such as an electrode, colloid particle, or the like. Those of ordinary skill in the art can select from among a wide variety of surfaces, functional groups, spacer moieties, etc. An exemplary description can be found in U.S. Pat. No. 5,620,850. This U.S. Patent also describes a variety of metal binding tags that can be used, including nitrilotriacetic acid, 2,2′-bis(salicylideneamino)-6,6′-demethyldiphenyl, and 1,8-bis(a-pyridyl)-3,6-dithiaoctane, or the like.

The term “self-assembled mixed monolayer” refers to a heterogeneous self-assembled monolayer, that is, one made up of a relatively ordered assembly of at least two different molecules.

The kits described herein, contain one or more containers or packages, which can contain, or otherwise hold together as a group, compounds such as the species, signaling entities, biomolecules, and/or particles as described. The kits also may contain instructions for mixing, diluting, and/or administrating the compounds. The kits also can include other containers with one or more solvents, surfactants, preservative and/or diluents (e.g. normal saline (0.9% NaCl, or 5% dextrose) as well as containers for mixing, diluting or administering the components to the sample or to the patient in need of such treatment.

The compounds in the kit may be provided as liquid solutions or as dried powders, or in essentially any other form compatible with the compounds. When the compound provided is a dry powder, the powder may be reconstituted by the addition of a suitable solvent, which also may be provided. Liquid forms of the compounds may be concentrated or ready to use. The solvent will depend on the compound and the mode of use or administration. Suitable solvents for are well known for drug compounds and are available in the literature.

A “therapeutic protocol” as described herein, refers to essentially any course of treatment, or subset or component thereof, known to be or proposed to be useful for the treatment and/or prevention of a disease state or medical condition of a patient (as contrasted with procedures involving merely analytical detection and/or diagnosis). Therapeutic protocols can involve, for example, administration of one or more drug agents or other compounds to a patient, or a sample derived from a patient, and can include aspects characterizing the protocol which can include, for example, the identity of any drug agent(s) administered, dosages, timing or sequence of administration, mode of administration, etc. “Effectiveness” or “efficacy” of a treatment protocol as described herein refers to the ability of the protocol to effect a desirable treatment and/or preventative outcome for a patient. Aspects of effectiveness or efficacy can include, but are not limited to, the ability of the treatment protocol to counteract, mitigate and/or prevent a disease state or medical condition and the presence or lack of undesirable effects of the treatment protocol, such as toxicity or deleterious interference with other treatment protocols indicated for a patient or other biological processes of a patient.

“Applying to a localized region of a body of a patient” as used herein involves application of an agent or material (e.g. a binding partner, signaling entity, colloid, etc.) to a discrete, predetermined area of a body. This is to be contrasted with application of an agent indiscriminately and/or systemically to or throughout the body (e.g. by injection into the vasculature of a patient for systemic circulation of the agent throughout the body, ingestion of the agent by the patient etc.). Some embodiments involving the application of an agent or material to a localized region of a body of a patient involve application of the material or agent to a surgical field or site on or within the body of a patient undergoing a surgical procedure. A “surgical procedure” as used herein can encompass essentially any invasive or minimally invasive procedure wherein tissue and/or cells of a patient are removed from and/or structurally altered within a localized region of a body of a patient. Such procedures can be performed internally of a patient (i.e. “within” a patient's body) and/or on an external surface of a patient (e.g. on the skin, eye, etc). Surgical procedures include open procedures as well as minimally invasive procedures such as those performed laproscopically or endoscopically.

“Promoting,” when used in the context of promoting an action (e.g. the application of a substance to a localized region of a body of a patient), refers to instructing, directing, encouraging, suggesting, etc. that the action be undertaken. Such promoting can take place, for example through any of a wide variety of means of communication between a person(s) or entity so promoting and a person(s) or entity to whom the promotion is directed. These include but are not limited to, verbal communications, written communication (e.g. letters, e-mail, publications, provision of written instructions—which may be provided as part of a kit, etc.), various forms of advertising, etc.

Certain aspects of the present invention are based, at least in pat, on and/or employ at least one interaction between chemical or biological agents for analysis, drug screening, or the like. The invention includes but is not limited to analyzing and/or inhibiting ligand interactions, including but not limited to ligands on intact cells (growing on an electrode, or in solution or in suspension, or within or on the body of a patient). The present invention contemplates a variety of embodiments including the use of drug candidates, known or putative ligands, and small molecule drug libraries.

In one embodiments for performing assays on a sample comprising cells, cells are grown on electrodes that may or may not be derivatized with self-assembled monolayers (SAMs). Putative ligands (e.g. for a particular cell-surface receptor) are immobilized on a solid support (e.g. gold colloids) along with signaling elements (e.g. electroactive complexes). These derivatized solid supports are incubated with the cells immobilized on a sensing electrode (e.g. metal support). The interaction between the target receptor and the ligand on the solid support (e.g. colloid-bound ligand) tethers the co-immobilized signaling elements near the sensing electrode. Compositions are then analyzed by alternating current voltammetry (ACV) or other electronic or electrochemical detection methods. As would be known by those of ordinary skill in the art, as used herein, “cell-surface receptor” is a generic term encompassing also cell surface proteins. Cells in solution, alternatively, can be attracted to a detecting electrode by electrophoresis. Specifically, cell-derived molecules can be bound to a ligand(s) that are attached to a colloid that also displays electro-active compounds such as ferrocene derivatives to aid in the detection of the bound complex. The use of electroactive complexes as signaling elements and the use of sensing electrode(s) as a substrate for cell immobilization/growth and/or for detection of binding interactions comprises one embodiment for performing certain assays of the present invention. These techniques are described in greater detail in international patent publication no. WO 00/43783, previously incorporated by reference.

In some preferred embodiments, interactions between a target receptor and ligand, or more generally between any two molecules capable of biologically binding to each other, can be determined via colloid aggregation resulting in a color change or change in visual contrast and/or optically or spectroscopically detectable signaling elements, such as fluorescent signaling elements as described briefly below and in greater detail in international patent publication nos. WO 00/43791 and WO 02/01230 and international patent application no. PCT/US01/44783, all previously incorporated by reference.

Certain embodiments of the invention make use of techniques for forming self-assembled monolayers on surfaces, and articles having surfaces coated with SAMs. These techniques have been previously described in international patent publication nos. WO 00/43783, WO 02/01230 and WO 00/43791, all previously incorporated by reference. Preferably, all of the species that participate in the SAM include a functionality that binds, optionally covalently, to the surface. In some embodiments a self-assembled monolayer is formed on a gold colloid. A self-assembled monolayer, whether formed on a colloid or on another surface, can be comprised of a mixture of thiol species (when gold is the surface) that can expose present (expose) essentially any chemical or biological functionality. For example, they can include tri-ethylene glycol-terminated thiols to resist non-specific adsorption and thiols terminating in a binding partner of an affinity tag, e.g. terminating in a chelate that can coordinate a metal such as nitrilo tri-acetic acid which, when in complex with nickel atoms, capture histidine-tagged binding species. The present invention employs techniques for rigorously controlling the concentration of essentially any chemical or biological species presented on a colloid surface or any other surface. Without this rigorous control over peptide density on each colloid particle, co-immobilized peptides would readily aggregate with each other to form micro-hydrophobic-domains that would catalyze colloid-colloid aggregation in the absence of aggregate-forming species present in a sample. This is an advantage of the present invention, over existing colloid agglutination assays. In many embodiments of the invention the self-assembled monolayer is formed on gold colloid particles. In some alternative embodiments, surfaces, particles, or colloids need not include SAM-coatings—essentially any known coating provided on a particle that provides a means for attaching a binding species and has capability to deliver a signal and/or provide a means for attaching a signaling entity can potentially be substituted.

The present invention also can employ assays involving colloid-colloid interaction. Some assays of the invention utilize first and second colloid particles that are allowed to become immobilized with respect to each other, or are prevented from becoming immobilized with respect to each other. Assays can indicate binding interactions, can involve enzymes that facilitate binding interactions, enzymes that cleave, drugs that inhibit binding interactions, or essentially any other binding interactions, the existence or lack thereof desirably being determined. Although this aspect of the invention is described with respect to first and second colloid particles becoming immobilized with respect to each other, generally many colloid particles would be involved in a particular assay, and observed to determine whether aggregation of the colloid particles, characteristic of binding between them or binding to a common surface, occurs. Where aggregation does not occur a solution in which colloid particles are suspended remains pink. Where aggregation does occur, the solution will become blue or purple, and in many cases a visible reticulum (visible aggregation) will result. The reticulum can be determined visibly with the human eye, or microscopically.

One advantage of this aspect of the invention is that color change, or lack thereof, can be determined spectroscopically. For example, a particular assay can be established for the determination of the ability of a candidate drug to inhibit binding between first and second species. The first and second species can be immobilized relative to (e.g., directly fastened to colloid particles), and the particles can be provided in separate containers (e.g., separate wells of a multi-well plate), and exposed to different candidate drugs. The wells can be measured spectroscopically for a change in absorption at a particular wavelength indicative of a color change resulting from colloid aggregation. Thus, one aspect of the invention involves automatically, via instrumentation, determining aggregation of colloid particles indicative of binding interaction or prevention thereof. In this and other assays species can first be immobilized relative to (e.g., fastened to) colloid particles and then exposed to a candidate drug, enzyme, or other species that may inhibit or facilitate binding, followed by exposure to the candidate drug or enzyme. Or first or second chemical or biological species can first be exposed to a candidate drug, enzyme, or the like followed by exposure to colloid particles to which the first and second chemical or biological species have the ability to fasten or become immobilized. Regardless of the order in which steps of assays of the invention are carried out, colloid-colloid aggregation is indicative of binding interactions or prevention thereof, and can be automated.

Signaling entities are used in a variety of assays and arrangements of the invention. It is to be understood that any of a variety of signaling entities can be selected in each case, including a dye, pigment, electroactive molecule, fluorescent moiety, up-regulating phosphor, enzyme-linked signaling moiety including horse radish peroxidase and alkaline phosphatase, chemiluminescent moiety, electrochemiluminescent moiety, etc. See for example, Knight, “Trends in Analytical Chemistry”, vol. 18, 1999, pg. 47; Knight, et al., Analyst, vol. 119, 1994, page 879; Stults, et al., “Use of Recombinant Biotinylated Aequorin in Microtiter and Membrane-Based Assays: Purification of Recombinant Apoaequorin from escheria coli”, Biochemistry, 1992, 31, 1433; Mengeling, et al., “A Microplate Assay for Analysis for Solution-Phase Glycosyltransferase Reactions: Determination of Kinetic Constants”, Analytical Biochemistry, 119, 286, (1991). A variety of signaling entities can be immobilized relative to surfaces of articles such as beads or colloid particles, if desired. Signaling entities presented on any of these can be fluorescent molecules. Fluorescent-conjugated antibodies and other fluorescent fusion proteins, including green fluorescent proteins, are widely used in biomedical research and testing. These fluorescent proteins and molecules can easily be attached to gold colloids that also present putative binding partners either through affinity tags, EDC/NHS chemistry or by binding to a His-tagged protein A or G presented on NTA-SAM-coated colloids according to the invention. Signaling entities such as fluorescent moieties also can be co-immobilized on a colloid via a biotin terminated ligand, or may be fastened via a chelate/metal/metal binding tag linkage. A fluorescent moiety may also be fastened by attaching it to an antibody and using a chelate/metal/metal binding tag with His-protein G to bind the antibody. The moieties can then be directly detected.

In some embodiments, the assays of the invention can make use of two or more distinguishable signaling elements, such as two ferrocene derivatives that oxidize at different potentials or two or more fluorescent moieties that absorb or emit electromagnetic radiation at different wavelengths from one another. In one example, a first ferrocene derivative or fluorescent moiety can be directly or indirectly attached to a ligand for a cell-surface receptor of interest. A second ferrocene derivative or fluorescent moiety is directly or indirectly attached to a second ligand that binds to a constitutively expressed cell surface receptor. In this way, the ratio of the two signals can be used to calibrate the level of induction of a cell-derived molecule (e.g. protein) that is induced by a physiological, environmental, or disease-associated change in expression. In an alternative embodiment utilizing signaling elements comprising metal complexes, this comparison of expression ratios can also be done by having both ligands carry the same metal complex but incubating separate aliquots of the sample on separate electrode pads.

One aspect of the invention involves determining colloid/colloid interactions, indicative of binding interactions between species immobilized with respect to colloids. The ability to form SAMs on colloids in accordance with the invention is one technique for linking species to colloids for such studies. Colloids can be linked to species desirably studied for their ability to bind to each other, such as biologically-relevant binding partners such as ligands and receptors, or can carry linked species that may have the ability to bind to a common entity, or which can each link to a species immobilized with respect to another colloid particle. This finds use in drug studies as well. Species for study can be linked to colloids by any technique described herein, e.g. metal binding tag/metal/chelate linkages. For example, metal binding tags such as histidine-tags can be attached to ligands and tagged putative binding partners can be incubated together with NTA/Ni(II) presenting colloids. A visible reticulum (aggregation visible by the human eye, by microscopy, etc.) will result if the two components are binding partners. Alternatively, the putative binding partners can be GST fusion proteins that would bind to glutathione presented on the colloid. Other linkers useful for attaching a binding species or other participant in assays of the invention to a surface include affinity tags. Affinity tags are well-known species used widely in biology, biochemistry, etc.

In one embodiment, compositions and methods can be used to detect target proteins and their interactions with other proteins, nucleic acids and small molecules. It is not meant that the invention be limited to studying interactions that involve proteins. The methods described herein can be applied to the detection of any two species interacting with each other. As described previously, gold colloids have the intrinsic optical property that they appear pink when dispersed in a homogeneous solution. However, if the colloids are forced into close proximity to each other, then the solution turns toward the blue end of the spectrum. Proteins can be attached to gold colloids by a variety of methods described herein. The assay need not be limited to the detection of direct interactions. Ligands attached to colloids may cause the colloids to be drawn close together when the ligands recognize a common target, which may be a complex of biomolecules rather than a single target molecule.

First and second chemical or biological species can be immobilized relative to first and second colloid particles respectively, and other chemical or biological species can be immobilized with respect to other colloid particles in such studies. In some cases the first and second chemical or biological species will be identical, i.e., a plurality of colloid particles will carry the same immobilized entities. In other cases entities will differ. Chemical or biological species can be fastened directly to colloid particles, e.g., by being covalently attached to a SAM-forming species that fastens to the particles, or can be immobilized relative to colloid particles, e.g., by being fastened to a colloid particle, via another colloid particle. For example, a chemical species is immobilized with respect to a colloid particle if it is fastened to another colloid particle that is itself fastened to the colloid particle.

The invention also anticipates mixing a drug candidate with colloids presenting molecules that either directly or indirectly bind to each other and detecting a diminution of the color change from pink to blue or a reduction in the extent of visible reticulum formation. Conversely, methods of the invention can be used to identify molecules that facilitate the binding of two molecules to each other, either directly or indirectly.

Regardless of which embodiment described is employed, the assays can be readily adapted to screen drug libraries for compounds that inhibit or promote certain binding interactions associated, for example, with a disease state. In one exemplary drug-screening assay, one can attach binding species to colloids (or other particle) and incubate with solutions containing binding partners, able to cross link and aggregate the colloids upon binding, and a drug candidate. The solutions may or may not be agitated. As the binding species are incorporated into aggregates upon binding to the binding partners, they bring the attached colloids close to each other, which causes the colloid solution to change color (e.g. from a pink suspension to a dark blue precipitate in a clear solution). This transition is clearly visible by eye. By absorption spectrophotometry, the peak at 569 nm degrades as the colloids aggregate.

A variety of studies involving colloids/colloid aggregation can be carried out in accordance with the invention. One set of assays makes use of the effect of an absorptive or emissive species, immobilized with respect to a colloid particle, by a second species that is immobilized with respect to a second colloid particle, brought into proximity or removed from proximity of the first colloid particle by binding, cleavage, or other interaction desirably studied in accordance with the invention. For example, a fluorescent molecule may be immobilized with respect to a first colloid particle and a chemical species having the ability to quench fluorescence of the fluorescent molecule, i.e., effect emission of the fluorescent molecule, can be provided on a second colloid particle. Then, first and second species immobilized with respect to the first and second colloid particles, if they bind to each other, will bring the first and second colloid particles into proximity with each other, causing quenching of the fluorescent molecule. If the first and second species immobilize with respect to the first and second colloid particle, each can bind to a common analyte, then presence of the analyte will cause quenching of fluorescence, and absence of the analyte will avoid quenching.

A drug candidate may be studied for competition with the analyte for binding of one of the species, or binding with one site on the analyte. In this case, the analyte may be provided as a known species. Presence of the drug candidate will thus inhibit immobilization of the first and second colloid particles relative to each other, thus will inhibit quenching. Alternative embodiments involve enhancing emission or shifting the wavelength of emission or absorption of a first molecule, by a second molecule on a second colloid particle.

This colloid/colloid aggregation technique can be used to identify the binding partners of drugs or proteins of interest. This can be accomplished by attaching the drug or protein to one set of colloids and possible binding partners to other sets of colloids and assaying for a binding interaction between the two sets of colloids. Once a biological target of a drug or protein has been identified, candidate drugs can be added to the assay in the presence of the colloid-attached binding partners to disrupt binding of the drug or protein to the cognate ligand, allowing identification of synthetic mimics of the drug or protein on the first set of colloids. This technique is very useful in identifying the biological target of orphan drugs or uncharacterized proteins for diagnostic or drug-screening purposes. This technique will also allow identification of synthetic replacements or “mimics” of currently used drugs that are expensive or difficult to produce.

Another embodiment in which colloid particles can be immobilized relative to each other in such assays involves colloid each being immobilized with respect to a common surface. The common surface can be a surface of another colloid particle presenting binding partners of species on the first colloid particles. The common surface can also be the surface of an article such as a membrane such as a nitrocellulose membrane, a chip surface, a surface of an article derivatized with an SAM, cell, tissue sample, surgical field, or the like. In preferred embodiments. the surface to which the colloid particles can bind includes binding sites at a high enough density so that if binding occurs (between species on the common surface and species on the colloid particles), the colloid particles will be brought into close enough proximity that detection (via color change characteristic of aggregation, quenching of fluorescence, or other property described herein) can occur.

The present invention can utilize methods involving the attachment of ligands (including but not limited to putative drug candidates or known drugs) to a surface that can be particle-like and interact them with cell-derived proteins, e.g. cell surface proteins, that can be attached to supports or left intact on cells in an effort to identify binding partners, determine their presence or absence, and quantify their levels. Specifically, intact cells that present cell surface receptors can be used as the first binding partner. Known or putative ligands attached directly or indirectly to signaling entities act as the second binding partner. Alternatively, cell-derived proteins can be bound to a surface, and their ligand or binding partner attached to a particle that has signaling capability, such as a colloid or derivatized colloid. Drug candidates or known drug agents can be added to facilitate drug screening through disruption of the interaction. This method is also very useful in diagnostics. A protein or antibody can be immobilized on a surface, and a sample suspected of containing a binding partner to this protein can be incubated with the sample to facilitate binding, and the rest of the sample can be washed away. A particle bearing a ligand to the binding partner and a signaling capability can be added. If the sample contains the binding partner, the particle will be indirectly bound to the surface to give a signal. The surfaces can be recruitable particles. Attachments can be direct or indirect, i.e., attachments can involve two entities becoming immobilized with respect to each other. Particles can display immobilized antibodies, e.g., histidine-tagged Protein G to which antibodies bind, and the antibodies can recognize a common ligand, or can contain proteins that recognize each other.

Advantages of the disclosed technology over existing methods such as ELISA, fluorescent labeling and SPR include: In the disclosed technology, there is no need for protein labeling; the protein is attached to a labeled component. Gold colloids can be pre-labeled with both: a) a signaling moiety; and b) a functional group for protein attachment. For example, self-assembled monolayers that present both NTA/Ni²⁺, to capture histidine-tagged proteins, and a signaling entity(s), for signaling, can be formed on the colloids. SAMs that incorporate carboxylic acid groups, for the chemical coupling (standard EDC/NHS chemistry) of unmodified proteins, can also be used. The technology is modular. Virtually any biological species can be co-immobilized on colloids with a signaling entity.

Identification of drug candidates can also be accomplished by using a competitive inhibition assay. Specifically, a drug candidate free in solution can be separately incubated with the composition. Competitive inhibition to the target cell-derived protein occurring by drug binding to receptor or ligand can be observed as a time or dosage dependent loss of detection signal.

In one embodiment, one can detect and quantitate cell surface proteins as follows: Histidine-tagged ligands that recognize cell surface receptors are attached to colloids that bear SAMs presenting both NTA (to capture His-tagged proteins) and signaling elements. These biospecific, signaling colloids are then incubated with cells presenting target receptors. A detectable signal, for example visible color change of the solution, a fluorescent emission, or a current peak, depending on the particular signaling/detection protocol employed, will result if ligands immobilized on signaling colloids bound to their cognate receptors on the cell surface. Antibodies that recognize the cell surface receptor can be attached to NTA-ferrocene bearing colloids that have first been bound with His-tagged protein A or G. Alternatively, an antibody can be attached directly to a colloid via a metal binding tag/metal/chelate linkage, where the metal binding tag is linked to the antibody. Techniques for linking a histidine tag to an antibody can be found in “Construction of the single-chain Fv from 196-14 antibody toward ovarian cancer-associated antigen CA125” Hashimoto, Y., Tanigawa, K., Nakashima, M., Sonoda, K., Ueda, T., Watanabe, T., and Imoto, T.: 1999, Biological and Pharmaceutical Bulletin, Vol 22: (10) 1068-1072.; “Human antibodies with sub-nanomolar affinities isolated from a large non-immunized phage display library”, Vaughan, T. J., Williams, A. J., Pritchard, K., Osbourn, J. K., Pope, A. R., Earnshaw, J. C. et al. 1996, Nature Biotechnology Vol 14 (3) p. 267.; “Expression and purification of single chain anti-HBx antibody in E. coli” Zhou G, lui K D, Sun H. C., Chen Y. H., Tang Z. Y., and Schroder C. H., 1997, vol. 123(11-12) pgs 609-13.

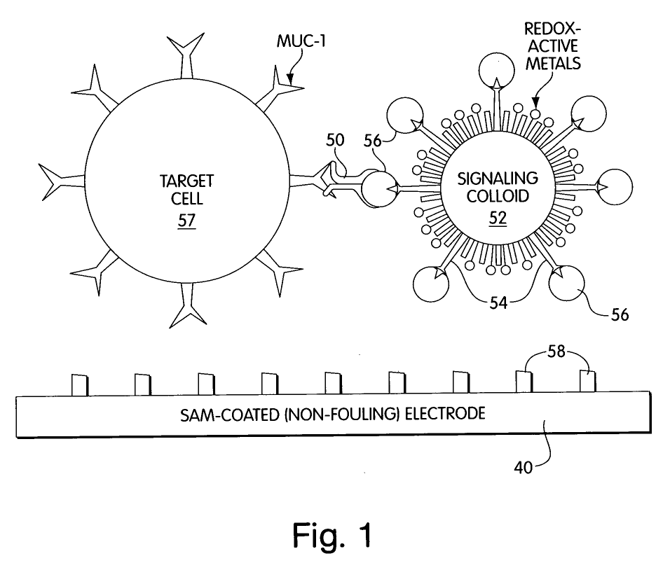

With reference to FIG. 1, an example of a useful technique involving fastening of a colloid particle to a cell is described. The tumor marker, MUC-1, is aberrantly expressed on neoplastic cells. The human tissue culture breast carcinoma cell line, MCF-7, available from the ATCC, over-expresses MUC-1. Antibody 50, DF3 or and DF3-p, available from the Dana-Farber Cancer Institute, is attached to electronic or electrochemical signaling colloids 52 (bearing NTA-SAMs 54) via a histidine-tagged protein G 56. Target cells 57 are incubated with the antibody-bearing signaling colloids 52, then electrophoresed to an electrode 40 coated with a SAM containing molecular wires 58 and analyzed by ACV. The SAM on electrode 40 includes molecular wires 58 admixed within more conventional, tight-packing SAM-forming species. For simplicity of illustration, only molecular wires 58 are shown schematically. A current peak results if the antibody-bearing signaling colloids are incubated with cells bearing MUC-1. Alternatively, a putative cognate ligand for MUC-1 can be His-tagged and attached to signaling colloids that also bear NTA groups.

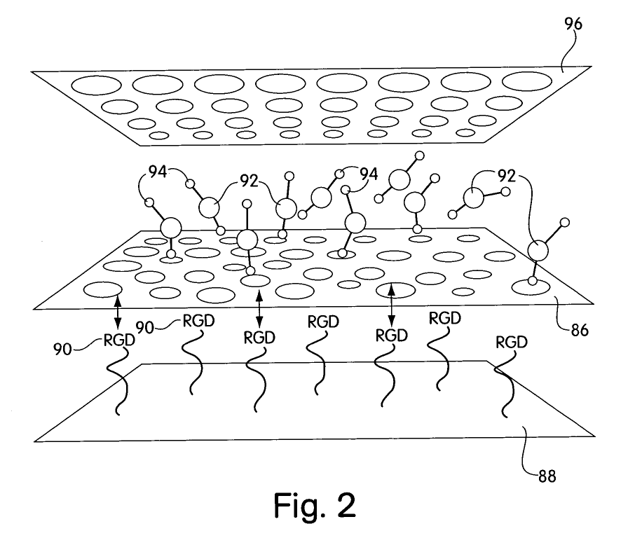

For embodiments of assays utilizing electronic detection of electroactive signaling entities, metallocenes are particularly useful as signaling entities for the following reasons. Various ferrocene derivatives and be selected to each oxidize at unique voltage between 100 mV to 800 mV. Each oxidation potential represents a unique label so that multiple cell surface targets can be simultaneously queried. If a biologically relevant interaction between a cell surface receptor and a colloid immobilized ligand occurs, the cell is decorated with electronic or electrochemical signaling particles and a current peak results. The magnitude of the current peak should be proportional to the number of cell surface receptors that were recognized by the signaling colloids. Cell-surface molecules can be detected on cells in suspension, in situ as in an operating field of a patient, or embedded in a tissue sample, as shown in FIG. 2. Frozen tumor specimens 86 are cryo-sectioned and placed directly onto a flexible, semi-permeable membrane support 88 that has been derivatized with cell-binding groups 90 such as RGD-containing peptides or methyl-terminated groups. The specimen is then incubated with electronic or electrochemical signaling colloids 92 that also present ligands 94 for a cell surface receptor of interest. Unbound colloids are washed away after an incubation period. The support membrane is then placed in physical contact with a microelectrode array 96, having electrode dimensions comparable to cell size, and analyzed by ACV. Each sector of the tissue specimen is analyzed for protein content and expression level, then correlated with histopathology. This capability ensures the relevance of single cell analysis because it enables the researcher to identify protein patterns that are associated specifically with cancer cells and discard random aberrant protein expression. Cells in suspension can be similarly attached to the support membrane.

Techniques described herein for determining binding of ligands or other binding species to cells can be used to identify cell-derived molecules, such as receptors or proteins, that are expressed differentially in healthy versus diseased tissue or cells. This differential expression can involve different levels of an expression in healthy versus diseased tissue or cells, and/or different patterns of expression on tissues or cells which can be readily identified. This technique facilitates diagnostic assays for determination of diseased states and in situ and intraoperative diagnostics, as described in greater detail below. For example, in connection with a patient suspected of having a particular disease, cells can be taken from the patient, specifically, cells that are associated with an indicator of the disease such as cells from a biopsy, blood sample, etc., and these cells can be analyzed versus healthy cells to determine expression levels or patterns indicative of disease. One can also use these techniques to screen for drugs that inhibit the upregulation of cell-derived proteins that are involved in various pathological conditions. Examples of two techniques for drug screening include: (1) administering a candidate drug to a patient suspected of or exhibiting symptoms of disease and monitoring a biological sample including cells of the patient as described above to determine efficacy of the drug in treatment of the disease; (2) taking a biological sample including cells from a patient suspected of having or having a disease and exposing the biological sample or components of the cells to candidate drugs, and monitoring expression level and/or pattern using the techniques described above. Once a binding partner (which can include a drug, antibody, or protein/peptide ligand for the cell derived protein) has been identified, the binding partner can be attached to a detection moiety to quantitate the expression level of the cell-derived protein in response to a disease state or a therapy. This can be any assay that tests for the indirect affects of drug candidates on the expression and translocation of cell-derived proteins both to the cell-surface and intracellular compartments.

The invention also provides the ability to visually investigate patterns of cell surface receptor expression on individual cell surfaces and/or on cells embedded in a tissue specimen or cells forming part of a tissue or organ of a patient, for example in a surgical field of a patient. This can be indicative of the pattern of cell surface receptor expression which can be correlated to a disease state. This can also be used in diagnostics or drug screening methods. In a particular assay, colloid particles carrying ligands that bind to cell surface receptors are exposed to individual cells or embedded cells or tissue(s)/organ(s) of a patient and the location of their binding with respect to individual cells can be determined visually, indicating the pattern of cell surface receptor expression. For example, MUC-1 is a cell surface receptor implicated in breast cancer. MUC-1 normally is expressed uniformly on surfaces of a variety of cell types. In transformed cells involved in a variety of cancers, the receptor is overexpressed and is concentrated at apical locations of the cell. This can be determined using the described technique. In drug screening, a culture of transformed cells can be provided and treated with drug candidates. The loss of the apical pattern expression is investigated. Visual identification, in this embodiment, can involve any technique described herein such as observation with the unaided human eye, microscopy, spectrophotometry, electron microscopy, fluorescence detection (including, by definition herein, phosphorescence detection), etc.

In the techniques involving electronic or electrochemical detection described above, the levels of expressed species can be compared between samples, including samples each involving an individual cell or other very small quantity, and patterns can be determined on larger samples including tissue samples. In connection with the visual detection embodiment described above, levels of expressed species can be determined as well as patterns of expressed species on both large samples and small samples including single-cell samples. Signaling entities useful in electronic or electrochemical detection include signaling entities described herein for electronic or electrochemical detection, including redox-active molecules such as ferrocenes. In connection with visual detection, any visual signaling entities described herein can be used including colloids, alone or carrying auxiliary signaling entities such as fluorescent or other visibly-identifiable entities. Multiple signaling entities can be used (i.e., multiple signaling per binding event). In connection with both electronic or electrochemical or visual signaling, different signaling entities can be used in connection with different assays. For example, a first ligand selected to target a first receptor or protein may be immobilized with respect to a first signaling entity while a second ligand, selected to target a second protein or receptor can be immobilized with respect to a second signaling entity. In electronic or electrochemical signaling the different signaling entities can include different redox potentials, the difference between which is distinguishable electronically, and in connection with visual identification different signaling entities can be different colors of emissive or absorptive entities. In such a case not only can expression level and pattern of proteins or receptors be determined but patterns can be differentiated in terms of location of expression of one receptor or protein versus another.

Customized Therapeutics and In Situ diagnostics:

In one aspect, the invention involves methods and techniques designed for patient-customized drug screening and therapeutic protocol screening. The methods can involve generally exposing at exposing at least a portion of a biological sample derived from a patient indicated for treatment for or at risk of acquiring a medical condition to a therapeutic protocol known to have efficacy for treating or preventing the condition, determining a response of the biological sample indicative of the effectiveness of the therapeutic protocol in treatment or prevention of the condition, and treatment of the patient according to the protocol. Typical embodiments can also involve exposing at least a portion of the biological sample a second and additional therapeutic protocols known to have efficacy for treating or preventing the condition, determining a response of the biological sample indicative of the effectiveness of these protocols in treatment or prevention of the condition, and treating the patient according to which of the above-tested therapeutic protocols showed the greatest efficacy in the determining steps.

In one embodiment, drugs are screened for their ability to target sites associated with a particular patient's physiology. In this embodiment, drugs are screened for their ability to block cell surface receptors or targeted protein-ligand interactions that are particular to a specific patient or group of patients that share common molecular profiles. Individuals have genetic variations that, although not severe enough to be termed mutations, result in slightly altered protein binding specificities. Therefore, to identify drugs and treatment protocols that are ideally suited to a particular patient or patient group, components of a standardized drug screening assay must be substituted with sample components derived from the patient. For example, in a drug screening assay, drugs are selected for their ability to disrupt the binding of protein X to protein Y. However, the assay produces several drugs that bind to the standardized targets with a gradation of affinities. In many cases, the efficacy of a drug is linked to its binding affinity. To determine which of these drugs is best suited for a particular patient, a secondary focused drug screen is performed, according to the invention, for example on samples of cells taken from a patient or samples derived from such cells, in which the sequence of proteins X and Y exactly match the patient's protein sequences. In this way, the treatment protocol can be optimized for the individual, who can be subsequently be treated according to the treatment protocol.

Drug screening assay that are compatible with methods of the invention are described above and in more detail in international patent publication nos. WO 00/43783 and WO 02/01230 and in international patent application no. PCT/US01/44783, all previously incorporated by reference as well as in International Patent Application no. PCT/US01/46221 by Bamdad et al., filed Nov. 15, 2001, entitled “Endostatin-Like Angiogenesis Inhibition,” incorporated herein by reference. In preferred embodiments, putative binding partners, such as proteins X and Y, are immobilized onto separate sets of SAM-coated gold colloids via a binding partner/affinity tag interaction. When the two sets of colloids are mixed together, inherent optical properties of the gold colloids cause the solution to turn from pink to blue if binding occurs. Disruption of the targeted interaction by a drug for example, causes the solution to revert to pink. To customize the drug screen, proteins identical to those of a particular individual are immobilized on the colloids, thus identifying drugs that are uniquely suited to disrupt that patient's targeted interaction.

In some cases, variations among individuals are more complex and may occur in a variety of molecules that take part in some aspect of the targeted mechanism, which can include several simultaneous or sequential protein-protein interactions. For these cases, components that are derived from the patient may be used to customize the drug screening assay or a secondary focused assay. For example, a drug screen for compounds to treat MUC 1-associated cancers identifies compounds that disrupt interactions between a defined receptor and uncharacterized ligands in a cell lysate (see international patent application no. PCT/US01/44783, previously incorporated by reference). A portion of the MUC1 receptor is immobilized on SAM-coated gold colloids and incubated with lysate and supernatants from MUC1+tumor cells. A ligand in the lysate acts to dimerize the colloid-immobilized receptor and the solution turns from pink to blue. Ligands of the receptor may include enzymes that modify the receptor or its ligand(s) as well as binding partners of the modified or unmodified receptor. Compounds that disrupt multimerization cause the solution to remain pink. To customize this complex assay, the tumor cell lysate/supernatant used in the standardized assay is replaced with the lysates/supernatants from a particular cancer patient to test different drugs or therapeutic protocols to identify the optimal drug or therapeutic protocol for that individual.

Further, therapeutic protocols may be tested for efficacy against tumor cells derived from the patient. Using convenient and generalizable colloid-based techniques described above and in more detail in international patent publication nos. WO 00/43783 and WO 02/01230 and in international patent application no. PCT/US01/44783, all previously incorporated by reference, the efficacy of treatment protocols can rapidly assessed by, for example, detecting levels of an expressed cancer marker, cell proliferation, etc.

In one embodiment, cells derived from a patient are exposed to colloid particles carrying immobilized binding partners of cell surface receptors in the presence of a candidate drug suspected of having the ability to block receptors (thereby reducing or eliminating colloid binding), or having the ability or suspected of having the ability for treatment of a particular medical condition that the patient experiences. The existence, level, and/or pattern of the particular receptor expressed by the patient's cells can first be determined in the absence of the drug or candidate drug, and then in the presence of the drug or candidate drug. Additional candidate drugs can then be screened in this way to determine their potential effectiveness for treatment of a particular medical condition in a particular patient. Drugs known to have at least some ability to treat a particular medical condition can be screened for a specific patient's receptiveness to the treatment. Drugs and treatment protocols which show the greatest efficacy in the screening tests can then be applied to the patient.

In one embodiment, colloids are exposed to a patient's cell to which candidate drugs have been exposed at at least two different points in time. For example, a patient's cell may be exposed to a candidate drug simultaneously with exposure to a colloid particle carrying a ligand for the receptor to which the drug may bind, or the colloid particle carrying the ligand may be added at any time, and in a second set a colloid particle carrying the ligand is added at a later point in time. For example, a cell can be exposed to the drug followed by exposure to a colloid particle carrying a ligand for the receptor at time X, and in a separate experiment, the same patient's cell can be exposed to the same drug and a colloid particle carrying a ligand for the receptor can be exposed to the system at a time X+Y. The result of this experiment can indicate whether the drug blocked the receptor for the period of time X+Y, or whether the drug is engulfed by the cell and expelled.

The above techniques involve in vitro as well as in situ experimentation. Techniques involving in vivo experimentation can be carried out as well, according to the invention. In one technique a patient is administered a drug or candidate drug and prior to, during, or after administration of the drug, a sample is drawn from the patient and an assay as described above is carried out. It can be very useful to carry out such assays both before and after treatment with the drug, to determine the activity of the drug, if any, in changing production of a particular species by the patient (which may correlate to effectiveness of the drug in treating a medical condition of the patient). For example, the patient's cell can first be assayed to determine the existence, level, or pattern of expression of a receptor or protein. Then the patient can be administered the drug (or another treatment protocol optionally involving the drug) and, following treatment, another of the patient's cells can be analyzed in a manner similar to analysis prior to treatment. Change in the existence, level or pattern of cell receptor or protein expression can indicate effectiveness of the particular treatment protocol in connection with the particular patient.

Cells can be taken from a patient at any of a variety of points in time and assayed to determine a change in essentially any material that they produce, typically a change in expression of a protein or receptor. The different points in time can correlate to a change in disease state of the patient, a change in treatment protocol (e.g. a change in drug protocol), or simply the passage of time during treatment with a single protocol.

Customized therapy of the invention can involve essentially any physical condition treatable with essentially any agent where the difference in effectiveness of at least two candidate treatment protocols, or the effectiveness of a single treatment protocol over a particular period of time, can be determined in an in vitro assay. Assays of the present invention can involve whole cells, lysate or plasma, or any other patient sample such as tissue, blood or other bodily fluid such as lymphatic fluid.

In another assay one drug is screened, or a variety of drugs are screened, for their effectiveness in binding to a particular cell receptor and in destroying the cell. This can have particular use in chemotherapy. Ideally, in chemotherapy, a drug will bind to a cancerous cell and kill it. But cancerous cells may produce mutant receptors to which drugs may not be able to bind. Finding a drug that can bind to a mutant receptor can be carried out using assays of the invention. Cancerous cells derived from a patient are exposed to candidate drugs, and colloids carrying immobilized binding partners of the candidate drugs are then exposed to the cells. Binding of the colloids to the cells can indicate (as compared to a control) binding of the candidate drug to the mutant receptor.

In another assay a variety drug candidates can be immobilized to a surface of a chip, ideally at separate, isolated locations. The chip can be exposed to a sample derived from a patient. Antibodies to the drugs, present in the sample, will bind to locations at which the drug is present. Then, exposure of the surface to colloid particles carrying immobilized binding partners of the antibodies (e.g., colloid particles carrying immobilized Protein G or Protein A), will bind to those regions.

Another aspect of the invention involves clinical diagnosis, selection of and evaluation of clinical treatment chemistry and/or protocols, and determination of the effects of treatment upon patients, using assays and combinations of assays of the invention. One general technique involves diagnosis. In this technique, a patient who may be healthy, or may be indicated for treatment for a particular medical condition, is tested as follows. A biological sample derived from the patient is exposed to an assay of the invention for determination of the existence, level, and/or pattern of a biological species indicative of a particular medical condition. In one embodiment, colloid particles that present a binding partner of the species are exposed to the sample or sample area, and it is determined whether the species indicative of a particular medical condition is present, in what amount, and/or in what pattern. Binding partners can be immobilized with respect to colloid particles for such assays in any manner previously described. In one embodiment, the binding partner carries an affinity tag that binds to a functionality that is attached to a SAM-forming species and incorporated into a SAM on a surface of the colloid. Attachment to the SAM-forming species can take place prior to, during, or after formation of the SAM on the colloid, and can involve affinity tag-involved linkage such as metal binding tag/metal/chelate linkage, EDC/NHS chemistry, or the like.

The value of any diagnostic is a measure of its sensitivity and practicality. Sensitivity is important for diagnosis of cancer and evaluation of complete surgical removal of affected areas. For enhanced sensitivity, in some embodiments, auxiliary signaling entities can attached to colloids carrying binding partners. In other embodiments, the colloid particles can act as the signaling entities. The use of colloids including auxiliary signaling entities can be advantageous in that a biospecific probe (e.g. binding partner) is attached to a particle which also presents a multitude of signaling entities (e.g. fluorescent moieties). A sample derived from a patient may be probed for the presence of several different disease-associated markers in a homogeneous assay as follows. A first biospecific probe that binds to a first marker is attached to a first set of colloids that bears moieties that fluoresce at a first wavelength. A second probe having a different specificity is attached to a second set of colloids that bears moieties that fluoresce at a second wavelength, and so on. The sample is bathed in the collection of colloids, then washed. The specimen surface is analyzed by optical detectors. The presence, intensity or ratio of one signal to another is assessed to determine a diagnosis.

Certain methods of the invention involve the use of signaling entities (e.g. colloids or colloids displaying auxiliary signaling entities), immobilized relative to biospecific probes (binding partners), as a diagnostic tool. The probes that are binding partners of the species indicative of a certain medical condition may include proteins, peptides, antibodies, small molecules, synthetic compounds, and natural products.

Samples used with assays of the invention may include samples taken from a patient, such as tissue specimens, cells, or bodily fluids. Samples may also involve samples derived from a patient such as cell lines derived from a patient's cells or agents or biological samples that have been stimulated by components such as antigens in a sample taken from a patient.

Where a whole cell is tested, assays can be carried out as described above in which immobilization of colloid to a cell surface can readily be determined visually or electronically or electrochemically, and determination can be made of the level of protein or receptor expressed at the cell surface. In some cases, the location or pattern of protein or receptor expression can be determined which, as discussed above, can be indicative of a particular medical condition. Cells or other samples can be assayed by attachment to a surface of an electrode and determination of whether colloid particles bind to the sample by incorporation of a redox-active or other electroactive species to colloids. Colorometric determinations can be carried out as well (facilitating determination of pattern of protein or receptor).

In one embodiment, a sample is derived from a patient and is assayed, using colloids carrying a binding partner for a component of the sample, in a variety of ways, to determine the existence of or level of the component in the sample. For example, endostatin has been used to treat a variety of cancers by inhibiting angiogenesis. Endostatin is a naturally occurring protein whose production appears to be modulated by a cancerous, primary tumor. Non-metastatic cancer patients with a primary tumor over-express endostatin. Removal of the primary tumor results in a drop in the production of endostatin and a subsequent increase in the emergence of distant metastases. Since typical treatment protocols for cancerous solid tumors begin with removal of the primary tumor, it was reasoned that metastasis may be kept in check by administering endostatin to the patient. At some point during treatment, the patient may develop antibodies to endostatin, or other therapeutic agent being administered, thus lessening the effectiveness of the therapeutic. Therefore it would be advantageous to determine whether antibodies to endostatin are present in the patient at a level high enough to hinder endostatin treatment (or for determination of the antibody for other purposes such as diagnosis of the disease). Endostatin can be immobilized on a surface of an article, and the surface can be exposed to a sample from the patient. If endostatin antibodies are present in the sample, then they will bind to endostatin immobilized at the surface. Then, typically following a rinse step, the surface can be exposed to colloid particles carrying an immobilized entity that will bind to endostatin antibodies, such as Protein A or Protein G. Immobilization of the colloid particles at the surface is then indicative of presence of antibody linked to endostatin immobilized at the surface. Identification of colloid particles immobilized at the surface can be carried out using techniques described herein.

Alternatively, the patient's cells may not express receptors to endostatin or may produce a mutant receptor. In such a case this treatment would not be advised for the patient. This applies to any of a variety of drugs, not limited to endostatin.

This technique can be used to determine a wide variety of analytes in a sample. Endostatin can be replaced by a binding partner of essentially any analyte, followed by exposure to colloid particles carrying an immobilized binding partner of the analyte. The surface upon which the assay takes place can be a chip, an electrode, magnetic bead, colloid particle (resulting in agglomeration where binding occurs), or other arrangement described herein. A multi-analyte assay can involve a chip including many regions at which different binding partners are immobilized. Exposure to a sample may result in binding of one, many, or no analytes at the regions of the surface. Exposure to the surface of colloids, carrying binding partners of the suspected analytes, results in binding at locations where analytes have bound to binding partners at the surface, indicative of presence of the analytes in the sample.

Techniques of the invention can be carried out in the presence of, optionally, an auxiliary sample derived from the patient, for example bodily fluid from the patient or components derived from the patient's bodily fluids. This can be important for indication of whether an auxiliary component produced by the patient may naturally interact with an interaction desirably determined in accordance with the invention. For example, a patient may produce, naturally, a component that would naturally interact with endostatin or angiostatin binding to a cell surface receptor. It is important, for determining the efficacy of a therapeutic protocol proposed for treatment of the patient, to determine whether this kind of interfering product exists in a patient's bodily fluid.

Diagnostic techniques of the invention can also be used to test a sample drawn or derived from a patient or tested in situ, for example during an invasive or minimally invasive procedure (e.g. a surgical procedure), to determine the extent or type of disease or the involvement of certain areas of tissue. Methods of the invention can be used to perform in situ hystopathology, by applying at least one of a colloid and a binding partner immobilized relative to a signaling entity to a localized region of a body of a patient, for example a surgical site, which enable determination of immobilization of the signaling entity to a target within the region that can guide a surgeon in assessing in real time the amount of tissue needed to be remove during a surgical or medical procedure. For example, in an exemplary embodiment, immediately or shortly after the excision of a tumor, the remaining tissue in the localized region of the patient from which the tumor was removed can be tested in situ for the presence of cancer markers by bathing the area with colloids bearing binding partners of the cancer markers. The presence of the cancer marker is determined by either direct visualization of the attached colloids (for example via a color change of the surface to which the colloids are attached, as previously described) or by detecting a signaling agent, such as a fluorescent moiety, that has been attached to the colloids. In some types of cancer, such as breast and prostate cancers, although it is critical to remove all infected tissue, unnecessarily aggressive surgery could significantly alter the patient's quality of life. In situ histopathology provides the patient with a better outcome by giving the physician test results in real time, which guides the surgical procedure so that the surgeon is better able to determine the amount and/or location of tissue needed to be removed.

This inventive method can be a substantial improvement over typical current standard procedures in which technology to test excised samples is so cumbersome that it is housed in central laboratories where samples, excised from the patient, are analyzed some time after the initial surgery. Second and third surgical procedures are typical to ensure complete removal of the affected areas. A major problem with the above-mentioned prior art procedure is that the surgeon is “working in the dark” in that she has no way of knowing whether she has left diseased tissue (e.g. cancerous cells) behind at the surgical site. The delay in time required analyze excised samples using the above-mentioned standard technique typically causes several time lapses between procedures which delays treatment protocols and also gives cancerous cells the opportunity to move away from the primary site and trigger distant metastases.

In situ histopathology, provided according to some embodiments of the invention, can be used with procedures within the body of a patient or performed on an external surface of a patient, e.g. on the skin, eye, etc. The inventive techniques can be utilized in open surgical procedures and can also be used with minimally invasive procedures such as laproscopic and endoscopic procedures, for example by using an optical detection apparatus associated with the laproscopic/endoscopic instrumentation.

The following examples and experiments illustrate particular embodiments of the present invention and are not to be construed as limiting the invention to any particular embodiment.

### EXAMPLES

For colloid preparation, 1.5 ml of commercially available gold colloid (Auro Dye) were pelleted by centrifugation in a microfuge on high for 10 minutes. The pellet was resuspended in 100 uL of the storage buffer (sodium citrate and tween-20). 100 uL of a dimethyl formamide (DMF) solution containing 90 uM nitrilo tri-acetic acid (NTA)-thiol, 90 uM ferrocene-thiol, and 500 uM carboxy-terminated thiol. Following a 3-hour incubation in the thiol solution, the colloids were pelleted and the supernatant discarded. They were then incubated in 100 uL of 400 uM tri-ethylene glycol-terminated thiol in DMF for 2 minutes at 55° C., 2 minutes at 37° C., 1 minute at 55° C., 2 minutes at 37° C., then room temperature for 10 minutes. The colloids were then pelleted and 100 ul of phosphate buffered saline (PBS) were added. The colloids were then diluted 1:1 with 180 uM NiSO4 in the colloid storage buffer. 100 uL of a His-tagged peptide at 100 uM in PBS were added to 100 uL of NTA-Ni(II) presenting colloids and incubated for 0.5 hours. To get rid of free, unattached peptide, the colloids were then pelleted and the supernatant discarded. The colloid pellet was then resuspended in 100 uL PBS.

### Example 1

### Cell Detection

This example demonstrates both the advantage of forming a SAM on a surface that includes a mixture including a molecular species that enhances electronic communication across the SAM by forming a defect in the SAM allowing fluid to which the surface is exposed to communicate electrically with the surface, and the utility of attachment of a colloid carrying immobilized signaling entity to a protein. The protein is in turn immobilized at a cell attached to the surface of an electrode presenting the SAM. The defect in this case is caused by bulk of the a SAM-incorporated molecule including phenyl rings.

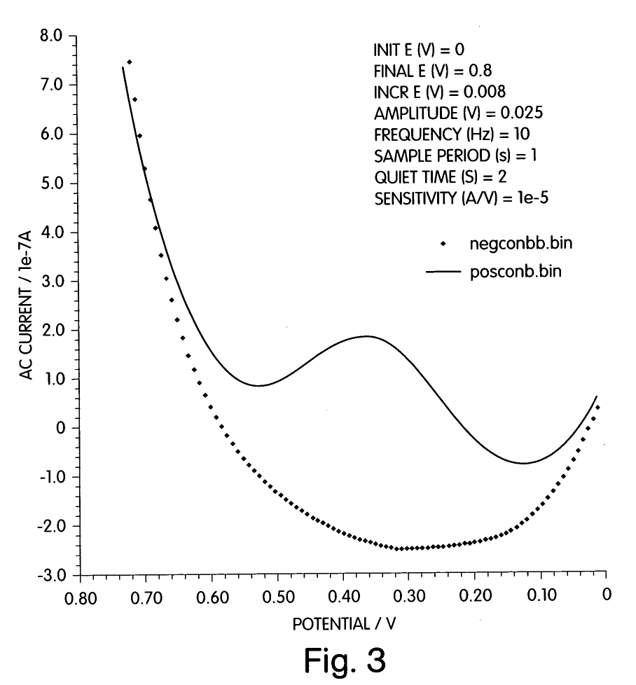

HUVEC cells were suspended in media and placed in a flask over a SAM coated on a gold surface. The SAM included 50% straight chain thiols, and 50% of the 2-unit poly (ethynylphenyl) thiol (MF1). 5 ul of an 8.4 mM RGD-His peptide solution was added to the media, and cells were incubated at 37C overnight to adhere to the electrode surfaces. After approximately 16 hours, 100 ul of SAM-coated colloids, displaying NTA for capturing the RGD-His peptide and ferrocene for signaling, were added to the cells and incubated for 20 min at room temperature. The electrodes were then rinsed in buffer to wash off any unbound colloids and measured. Current peaks were recorded at 220-250 mV. Negative controls were cells incubated with His-GST, an irrelevant protein that should not bind to cells. Colloids were added to negative controls, electrodes were rinsed in buffer, and measurements were taken. No peaks were observed for negative controls. FIG. 3 shows a peak (solid line) generated when colloid presenting ferrocene signaling entity and his-tagged ligand to a cell surface receptor is brought to an electrode surface by cell/surface interaction. Diamond represent negative control where colloids displayed an irrelevant protein selected not to bind to the cell surface receptor.

### Example 2

### Visual Detection of Colloid-Immobilized Ligand Interaction with Cell Surface Receptors and Disruption Thereof

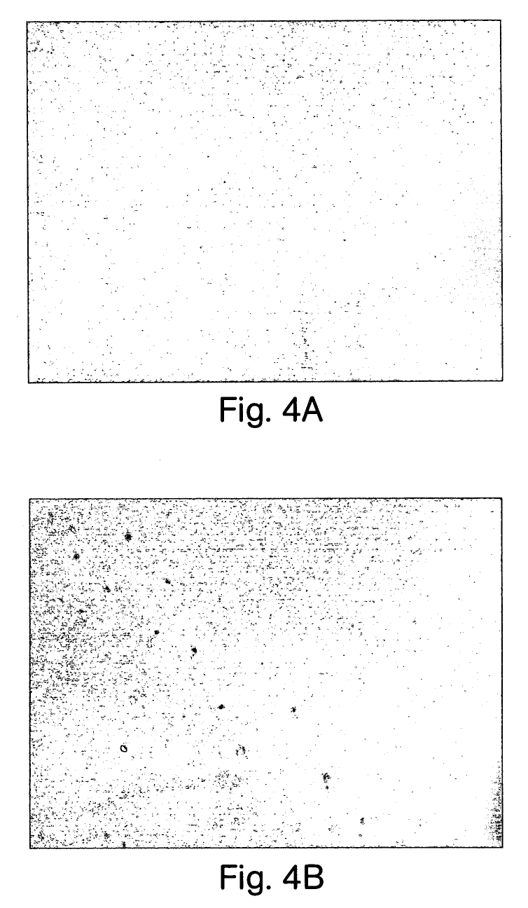

This example was carried out in a manner as described in Example 3 with the following exceptions. Cells were grown on a multi-well plate. Following interaction, visual inspection showed decoration of the cells with colloids selectively at locations on the cell where the receptor was expressed. Referring to FIG. 4A, a control is shown in which no binding occurred. A random sequence peptide was used. FIG. 4B shows decoration of cells with colloids selectively at locations on the cells where protein was expressed.

### Prophetic Example 1

### Customized Drug Screen

This example describes one way in which assays of the invention are used to predict a patient's response to a proposed treatment before treating the patient and choosing a preferred treatment protocol based on response indicators. Drugs are tested for activity in an assay that uses key responsive components that are particular to that patient.

A patient has been diagnosed with MUC1+breast cancer. An effective treatment strategy is to block the interaction of a portion of the MUC1 receptor with its cognate ligands, which results in inhibition of MUC1+cell proliferation. Suppose a number of drugs are available for use against the disease and that these drugs have all demonstrated efficacy in a functional MUC1+cell proliferation assay. To determine which of these possible therapeutics is best suited for a particular patient, they are tested screened against components that are particular to that patient.

Specifically, a histidine-tagged peptide corresponding (in sequence also identical to that of the particular patient) to the functional portion of the MUC1 receptor (see international patent application no. PCT/US01/44783, previously incorporated by reference) is immobilized on NTA-Ni2+-SAM-coated gold colloids. To the MUC1-presenting colloids are added a lysates/supernatants mixture that contains the activating ligands of the MUC1 receptor. The ligands of the MUC1 receptor dimerize or multimerize the receptor, which causes the attached colloids to be drawn close together. Due to an inherent optical property of gold colloids, when they are homogeneously dispersed in solution, the solution appears pink but when they are drawn close together, the solution turns blue. A drug candidate is added to the assay. Solutions that contain a drug that interferes with the MUC1-ligand interaction will remain pink and not undergo the color change.

In a non-customized drug screen, drugs were chosen using lysates/supernatants from MUC1+breast tumor cells (T47Ds) that originated from a cell line that was derived from one breast cancer patient. In contrast, in this customized drug screen, this “standard” lysate/supernatant is replaced by the current patient's lysate/supernatant. There are a multitude of factors in these lysates/supernatants that could vary from one patient to another that would have a large impact on the activity of a drug. For example, the mixtures contain enzymes that modify the receptor or ligand or both. Genetic variations in these enzymes would alter how the receptor/ligands were modified and this would affect the ability of the candidate drugs to recognize either binding partner. In other instances, a patient's disease state would affect levels of certain ligands or messengers that are key elements in the standardized drug screen.

Drugs are chosen from the panel of drugs recommended for treatment of the disease based on their patient-specific profile of drug activity. That is to say that drugs are selected based on the response of something in the assay that is particular to that the current patient upon exposure to the drug.

### Prophetic Example 2

### Individualized Whole Cell Drug Screen

This example describes how methods of the invention are used to identify which drugs from a candidate group are best for the treatment of a particular patient in terms of efficacy or toxicity. A cell line is generated from tumor cells drawn from the current patient. The patient suffers from a type of cancer for which there is a defined tumor marker whose density on the cell surface is proportional to disease stage and prognosis.

In this example, the patient has metastatic melanoma and the tumor marker is the cell surface receptor αVβ3. The patient's cells are expanded and aliquoted into 96-well plates. Vitronectin (of sequence identical to the current patient to account for possible effects of single nucleotide polymorphisms (SNPs)), which is the binding partner of the αVβ3 receptor, is expressed with a histidine tag and bound to colloids that are derivatized with SAMs that present both NTA-Ni2+ and biotin. The colloids are mixed with the patient's cells and binding between vitronectin and αVβ3 causes the attachment of colloids to the cells in the pattern and in an amount that reflects the position and density of the αVβ3receptors. Unbound colloids are rinsed away. Bound colloids are sensitively visualized by adding fluorescently labeled streptavidin and detecting using standard techniques, such as a fluorescence microscope.

To discriminate efficacy among a panel of candidate drugs against the patient's own cells, or to determine dosage levels, a drug is added to each aliquot of cells before colloid addition. The drug/cell mixtures are allowed to incubate for some period of time under a variety of conditions. At the end of the trial period, the probe colloids are added to the cells, rinsed, fluorescently labeled, then visualized. An effective treatment is determined based on the response of the patient's cells to the treatment.

In some cases, it may be desirable to use a patient's cells prior to immortalization. The techniques described are compatible with this approach and can be modified by using other signaling entities such as electroactive or electrochemiluminescent moieties to achieve the desired level of sensitivity.

While several embodiments of the invention have been described and illustrated herein, those of ordinary skill in the art will readily envision a variety of other means, techniques, compositions, articles and structures for performing the functions and/or obtaining the results or advantages described herein, and each of such variations or modifications is deemed to be within the scope of the present invention. More generally, those skilled in the art would readily appreciate that all parameters, dimensions, materials, configurations, etc. described herein are meant to be exemplary and that actual parameters, dimensions, materials, configurations, etc. will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. In the claims, all transitional phrases or phrases of inclusion, such as “comprising,” “including,” “carrying,” “having,” “containing,” “composed of,” “made of,” “formed of” and the like are to be understood to be open-ended, i.e. to mean “including but not limited to.” Only the transitional phrases or phrases of inclusion “consisting of” and “consisting essentially of” are to be interpreted as closed or semi-closed phrases, respectively.

What is claimed is:

1. A method, comprising:
applying to a localized region of a body of a patient a binding partner immobilized relative to or able to be immobilized relative to a signaling entity; and determining immobilization of the signaling entity within or on the body of the patient.

2-6. (canceled)

7. A method as in claim 1, wherein the patient is undergoing surgical procedure.

8-9. (canceled)

10. A method as in claim 7, wherein the surgical procedure is a minimally-invasive procedure.

11. A method as in claim 7, wherein the surgical procedure is an endoscopic or laproscopic procedure.

12. A method as in claim 7, wherein the surgical procedure involves removal of a diseased tissue.

13. A method as in claim 12, wherein the diseased tissue comprises a cancerous cell.

14. A method as in claim 12, wherein the diseased tissue comprises a tumor.

15-21. (canceled)

22. A method as in claim 1, wherein the binding partner is a biological binding partner.

23. (canceled)

24. A method as in claim 1, wherein the binding partner is an antibody.

25-28. (canceled)

29. A method as in claim 1, wherein the signaling entity is able to absorb or emit electromagnetic radiation.

30. A method as in claim 1, wherein the signaling entity is electronically detectable.

31. A method as in claim 1, wherein the signaling entity is chemically detectable.

32. A method as in claim 29, wherein the signaling entity is fluorescent.

33-38. (canceled)

39. A method as in claim 1, wherein the determining step comprises optically detecting the signaling entity.

40-67. (canceled)

68. A method as in claim 1 wherein an agglomeration of colloid particles acts as a signaling entity.

69-114. (canceled)

115. A method of claim 1, wherein the binding partner is a chemical compound.

116. A method of claim 1, wherein the binding partner is specific for a cell receptor.

117. A method of claim 1, wherein the cell receptor is MUC1.

118. A method of claim 24, wherein the antibody is specific for a cell receptor.

119. A method of claim 118, wherein the cell receptor is MUC1
